(19) United States
(12) Patent Application Publication
(10) Pub. No.: US 2025/0235455 A1
(43) Pub. Date: Jul. 24, 2025

(54) AQUEOUS TICAGRELOR SOLUTIONS, METHOD OF MANUFACTURING AND USES

(71) Applicant: HYLORIS DEVELOPMENTS SA, Liège (BE)

(72) Inventor: Atul PATIL, Liège (BE)

(21) Appl. No.: 18/855,499

(22) PCT Filed: May 16, 2023
(86) PCT No.: PCT/EP2023/063071

§ 371 Date: Oct. 9, 2024

(30) Foreign Application Priority Data
May 16, 2022 (WO) PCT/EP2022/063185
Mar. 7, 2023 (WO) PCT/EP2023/055736

(51) Int. Cl. A61K 31/519; A61K 9/00; A61K 9/08; A61K 47/22; A61K 47/40

(52) U.S. Cl. CPC A61K 31/519; A61K 9/0019; A61K 9/08; A61K 47/22; A61K 47/40

(57) ABSTRACT

The present invention provides an aqueous ticagrelor composition with improved storage stability comprising ticagrelor as active ingredient and a solubilizer for ticagrelor, preferably a cyclodextrin or vitamin E TPGS. The aqueous ticagrelor solution is preferably provided for intravenous administration, either by injection or infusion. Preferably an aqueous ticagrelor intravenous composition according to the invention, in a ready-to-use form or suitable for dilution prior to intravenous administration, is used as a medicine in the treatment of a ticagrelor responsive disease.

### TECHNICAL FIELD

The present invention is situated in the field of pharmaceutical compositions and medical uses of pharmaceutical compositions. The present invention also relates to the manufacturing of pharmaceutical compositions and unit dose forms. The active ingredient concerned is ticagrelor. The invention is advantageous as it provides stable ticagrelor iv formulations, where hereto only ticagrelor tablets are available. In addition, the invention provides aqueous ticagrelor solutions with an improved stability that is relevant for use in pharmaceutical commerce. The invention has for effect that patients with a ticagrelor-responsive disease can be treated effectively even if unconscious or even if having problems swallowing. Ready-to-use liquid ticagrelor formulations as provided by the invention avoid time needed to crush tablets and dissolve powder. This is especially important in emergency situations where an urgent treatment is required. It also allows for improved dose titration.

### BACKGROUND

Ticagrelor is a well-known active ingredient. It is a platelet aggregation inhibitor used for the prevention of thrombotic events, such as myocardial infarctions or strokes, in patients with acute coronary syndromes. Its chemical name is (1S,2S,3R,5S)-3-{7-[(1R,2S)-2-(3,4-difluorophenyl)cyclopropylamino]5-(propylthio)-3H-(1,2,3) triazolo (4,5-D) pyrimidin-3-yl}-5-(2 hydroxyethoxy)cyclopentane-1,2-diol.

Ticagrelor was developed by AstraZeneca and was approved for entry on the market by the European Medicines Agency in 2010 and by the US Food and Drug Administration in 2011. As drug it is marketed in tablet form under the name Brilinta® in the USA and Brilique® in the EU. It is not commercially available in liquid form. Ticagrelor is an oral, reversible, direct-acting P2Y₁₂ receptor antagonist that works by inhibiting platelet activation. Brilinta® tablets, together with aspirin, have shown to significantly reduce the risk of major adverse cardiovascular (CV) events (heart attack, stroke or CV death), in patients with acute coronary syndrome (ACS) or a history of heart attack. In the US, Brilinta® tablets are also indicated for the reduction of the risk of a first heart attack or stroke in high-risk patients with coronary artery disease.

Ticagrelor has shown to be highly susceptible to degradation when exposed to light, heat and oxygen, plus its limited solubility is a great challenge in formulating it as an aqueous solution. Although the need for a liquid ticagrelor formulation remains high, to our knowledge a successful commercial product of desired solubility and long-term stability in correspondence with requirements of the pharmaceutical industry has not been successful.

Sigfridsson et al. (J Pharm Sci 100:2194-2202, 2011) disclosed a composition deemed suitable for intravenous administration. The composition is based on nanoparticles of ticagrelor, a combination of polyvinylpyrrolidone and the disodium salt of Aerosol AOT for stabilization of the active ingredient, and 5 percent mannitol to obtain a nanosuspension. Aerosol AOT is believed to correspond to dioctyl sulfosuccinate sodium salt. Although the nanosuspension is reported to have a stability of at least 10 months, it is also reported that there is some tendency for particle aggregation and sedimentation during storage. Therefor, samples are sonicated prior to an intravenous administration. This is cumbersome for a pharmaceutical use and presents a safety risk.

The same publication of Sigfridsson et al. also mentions that ticagrelor concentrations in phosphate buffered solution at pH 7.4 declined after 1 month under normal laboratory conditions of light and temperature. Thus, the investigation of a solution as alternative to a suspension, is counter-indicated.

In the product characteristics for the medicinal product Brilique 60 mg film-coated tablets (Brilique INN-ticagrelor) it is disclosed that tablets can be crushed, mixed with water and drunk immediately, for patients who are unable to swallow the tablets. Alternatively, the mixture may be administered via a nasogastric tube into the stomach. The disadvantage of this formulation is that it is not readily available to patients. A preparation is needed just prior before administration. The formulation does not have a long-term storage stability and the tablet particles settle on standing. For emergency use, especially when a patient is unconscious, this is not a solution.

Cheong-Weon Cho et al., 2019 studied self-micro emulsifying drug delivery systems (SMEDDS) for oral delivery to overcome the poor ticagrelor solubility barriers. Ticagrelor solubility was studied in oily and hydrophilic excipients. A combination of surfactants was selected to obtain an emulsion system: Capmul MCM/Cremophor EL/Transcutol P. Ticagrelor is used to inhibit platelet aggregation in patients with acute coronary syndrome, but its poor solubility and low bioavailability limit its in vivo efficacy.

Yaye et al., 2014 studied the degradation of ticagrelor when exposed to heat, pH, peroxide and light. They identified numerous degradants DP1 to DP9 indicating that the molecule is highly suseptible to degradation.

In view of the above, there remains a need in the art for storage stable ticagrelor formulations with good ticagrelor solubility, particularly for emergency use. The objective of the present invention is to solve at least one or more problems as described above. In particular, the invention aims to provide a formulation comprising ticagrelor as active ingredient that has improved solubility in combination with long-term stability. Ticagrelor should be readily available to patients and the formulation developed should make use of ingredients that regulatory agencies find acceptable (e.g. within the FDA's Inactive Ingredient Guide—IIG limits).

### SUMMARY OF THE INVENTION

In a first aspect, the invention provides an aqueous ticagrelor solution, comprising ticagrelor and a solubilizer for ticagrelor with a storage stability of at least 3 months, at 40° C. and 75% Relative Humidity or at 25° C. and 60% Relative Humidity.

Preferably said solubilizer is a cyclodextrin or vitamin E TPGS.

Preferably the aqueous pharmaceutical ticagrelor solution according to an embodiment of the invention, comprises an inclusion complex of ticagrelor in a cyclodextrin, wherein the aqueous pharmaceutical ticagrelor solution comprises
0.10-14.0 mg/ml ticagrelor and
20-100 mg/ml of cyclodextrin in a quantity for solubilization of the ticagrelor in the selected volume of aqueous pharmaceutical solution (solubilizer for ticagrelor),
wherein the composition has a pH between 5.5-9 endpoints included,
and the aqueous pharmaceutical solution has a volume of 25 to 1000 ml,
with a storage stability of at least 3 months at 25° C. and 60% Relative Humidity.

Preferably the osmolality is between 300-900 mOsm/kg.

Preferably the cyclodextrin is selected from a hydroxypropyl-beta-cyclodextrin and a sulfobutylether beta-cyclodextrin.

Preferably organic co-solvents are excluded.

In a further aspect, the invention provides an aqueous pharmaceutical ticagrelor solution for use as a medicine. Preferably the composition is administered as an infusion.

Preferably said medicine is for use in the treatment of acute coronary syndrome (ACS), myocardial infarction (MI), ischemic stroke, transient ischemic attack (TIA) or for the reduction of platelet-tumor cell interactions in a patient in need thereof.

Preferably said medicine is for use in a dual antiplatelet therapy (DAPT), preferably comprising acetyl salicylic acid or salts thereof as second active ingredient in addition to ticagrelor.

In a further aspect, the invention provides a ready-to-use infusion container comprising a composition according to an embodiment of the invention.

Preferably the ready-to-use infusion container according to an embodiment of the invention, comprises 3000-16000 mg of cyclodextrin.

Preferably the ready-to-use infusion container according to an embodiment of the invention, comprises 5 w/v % dextrose or 0.9 w/v % sodium chloride.

Preferably the container is a bag or bottle.

In a further aspect, the invention provides, a method for the manufacturing of an aqueous pharmaceutical ticagrelor composition according to an embodiment of the invention, comprising the steps of:
preparing an aqueous solution of pH 5.5-9 preferably comprising a buffering agent, more preferably comprising a phosphate buffer,
introducing a cyclodextrin amount for inclusion of a pre-determined amount of ticagrelor (solubilizer for ticagrelor),
adding the pre-determined amount of ticagrelor, thereby obtaining an inclusion complex of ticagrelor in cyclodextrin.

Preferably heating is applied prior to the addition of ticagrelor.

Preferably the ticagrelor has a D90 particle size below 10 micrometers when tested using Malvern mastersizer.

Alternatively, an aqueous ticagrelor composition according to an embodiment of the invention comprises ticagrelor as active ingredient,
characterized, in that the composition is a solution comprising
a water-soluble inclusion complex of ticagrelor in a cyclodextrin,
wherein the composition has a pH between 6 to 8;
with a storage stability of at least 3 months at 40° C. and 75% Relative Humidity.

Preferably, the alternative aqueous ticagrelor composition has an osmolality between 350-900 mOsm/kg.

Preferably, the cyclodextrin in the alternative aqueous ticagrelor composition is selected from hydroxypropyl-beta-cyclodextrin and a sulfobutylether derivative of a beta-cyclodextrin.

Preferably, the alternative aqueous ticagrelor composition comprises 15-40% w/w cyclodextrin; preferably comprising 15-40% w/w hydroxypropyl-beta-cyclodextrin.

Preferably, the alternative aqueous ticagrelor composition has 2-15 mg/ml ticagrelor.

Preferably, the alternative aqueous ticagrelor composition, is provided for infusion and has a volume of 15 to 30 ml.

Preferably, the alternative aqueous ticagrelor composition is provided for injection and has a volume of 5 to 15 ml.

Preferably, the alternative aqueous ticagrelor composition, is with the proviso that organic co-solvents are excluded. Preferably polyethylene glycol is excluded from the aqueous ticagrelor composition.

Preferably the ticagrelor has a D90 particle size below 10 micrometer when tested using Malvern mastersizer.

More preferably, the alternative aqueous ticagrelor composition according to an embodiment of the invention consists of
5-15 mg/ml ticagrelor,
15-40% w/w of a hydroxypropyl-beta-cyclodextrin,
5 mM-20 mM of phosphate buffer,
optionally including a tonicity modifier,
wherein the pH is between 5.5 and 8.

In a further aspect, the alternative aqueous ticagrelor composition according to an embodiment of the invention is for use in a ticagrelor responsive medical treatment; preferably for use in a ticagrelor responsive medical treatment wherein the composition is administered as an injection or infusion, nasal gastric fluid, or drink.

Preferably, the alternative aqueous ticagrelor composition according to an embodiment of the invention, is for use in the treatment of acute coronary syndrome (ACS) or myocardial infarction (MI), ischemic stroke, transient ischemic attack (TIA) or for the reduction of platelet-tumor cell interactions in a patient in need thereof.

Preferably, the alternative aqueous ticagrelor composition according to an embodiment of the invention, is for use in a dual antiplatelet therapy (DAPT), preferably comprising acetyl salicylic acid or salts thereof as second active ingredient in addition to ticagrelor.

In a further aspect, the invention provides a unit dose composition for delivery of 50-180 mg ticagrelor comprising an alternative aqueous ticagrelor composition according to an embodiment of the invention.

Preferably said unit dose composition, comprises 2000-4000 mg of cyclodextrin.

Preferably said unit dose comprising, comprises 5% dextrose water as diluent.

In a further aspect, the invention provides a method for the manufacturing of an aqueous ticagrelor composition according to an embodiment of the invention, comprising the steps of:
preparing an aqueous solution of pH 5.5-9, preferably comprising a buffering agent,
introducing a cyclodextrin amount for inclusion of a pre-determined amount of ticagrelor,
adding the pre-determined amount of ticagrelor, thereby obtaining an inclusion complex of ticagrelor in cyclodextrin.

Preferably heating is applied prior to the addition of ticagrelor.

In a further aspect, the invention provides an aqueous ticagrelor solution for intravenous administration, for use in the treatment of acute coronary syndrome (ACS), myocardial infarction (MI), ischemic stroke, transient ischemic attack (TIA) or for the reduction of platelet-tumor cell interactions in a patient in need thereof.

In a further aspect, the invention provides an aqueous ticagrelor solution for intravenous administration, for use in a dual antiplatelet therapy (DAPT), preferably comprising acetyl salicylic acid or salts thereof as second active ingredient in addition to ticagrelor.

Preferably said aqueous ticagrelor solution for intravenous administration for use as a medicine according to an embodiment of the invention, has a storage stability of at least 3 months in accelerated storage conditions at 40° C. and 75% Relative Humidity.

### DETAILED DESCRIPTION OF THE INVENTION

The invention provides a solution to the problem of obtaining clear and improved storage stable aqueous ticagrelor formulations. The ticagrelor formulations of the invention are suitable for administration as an infusion. The cyclodextrin ingredients are acceptable for medical use according to the Inactive Ingredient Guide, also called the IIG-list. This contains inactive ingredients and the amounts that have been reviewed and approved by list the Food and Drug Administration for a dosage form and/or a particular route of administration. D-alpha-tocopherol polyethylene glycol succinate, known as vitamin E TPGS, has been approved by the Food and Drug Administration (FDA) as a safe adjuvant.

Unless otherwise defined, all terms used in the description of the invention, including technical and scientific terms, have the meaning as commonly understood by a person skilled in the art to which the invention pertains. Furthermore, definitions of the terms are included to better understand the description of the present invention.

As used here, the following terms have the following meaning: “A”, “an”, and “the” as used here refer to both the singular and the plural, unless the context indicates otherwise. “A surfactant” refers, by way of example, to one or more than one surfactant.

“About” as used herein, referring to a measurable value such as a parameter, an amount, a duration and the like, is intended to include variations of plus or minus 10% or less, preferably plus or minus 5% or less, more preferably plus or minus 3% or less, even more preferably plus or minus 1% or less, and even more preferably plus or minus 0.1% or less of the specified value, as far as such variations are suitable for carrying out in the described invention. It will be clear, however, that the value to which the term “about” relates is itself also specifically described. “Include”, “comprising” and “comprises” are used herein are inclusive or open terms that specify the presence of what follows, e.g. a component and the presence of additional, unnamed components, features, elements, parts, steps, which are well known in the art or described therein, and do not exclude them.

The recitation of numerical ranges by endpoints includes all numbers and fractions that are included within that range, as well as the endpoints mentioned.

The term “% w/w” as used herein means percentage by weight in which the weight ratio of an ingredient to the total weight of a composition is expressed as a percentage.

In a first aspect, the invention provides an aqueous ticagrelor solution, comprising ticagrelor and a solubilizer for ticagrelor with a storage stability of at least 3 months at 40° C. and 75% Relative Humidity or at 25° C. and 60% Relative Humidity. Most preferably the solubilizer is a cyclodextrin or vitamin E TPGS.

Preferably, the invention provides an aqueous ticagrelor composition comprising ticagrelor as active ingredient, characterized, in that the composition is a solution comprising a water-soluble inclusion complex of ticagrelor in a cyclodextrin, wherein the composition has a pH between 5.5 to 9; preferably 5.8 to 8.5; more preferably 6.0 to 8.2; even more preferably 6.2 to 8.1; most preferably 7.0 to 8.0. The pH range of 7.0 to 8.0 is especially important because of the physiological acceptance for iv formulation to patients.

By the term “ticagrelor” as used herein, is meant ticagrelor in free form as well as to its pharmaceutically acceptable solvates, hydrates, enantiomers, polymorphs, or mixtures thereof. Preferably ticagrelor is used in its free form.

Ticagrelor has the following chemical structure:

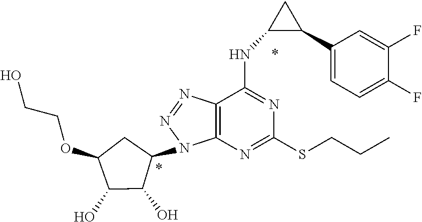 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor has six stereocenters and consequently there are many crystalline and amorphous forms. In a preferred embodiment of the present invention, the active agent is crystalline ticagrelor. Especially, four non-solvated crystalline forms are available, named as Polymorph I, II, III and IV. The polymorphs present different physical and chemical properties. In a preferred embodiment, polymorph II is used. Its X-ray powder diffraction patterns is characterized by specific peaks at 5.5° (±0.1°), 6.8° (±0.1°), 10.6° (±0.1°), 13.5° (±0.1°), 14.9° (±0.1°), 18.3° (±0.1°), 19.2° (±0.1°, 22.7° (±0.1°), 24.3° (±0.1°) and 27.G (±0.1°) 2Q. The polymorph II form is described in literature as the most stable crystalline form of ticagrelor.

Ticagrelor has a low and pH-independent solubility in aqueous media. This property of not ionizing in the physiological pH range, makes the development of liquid formulations especially challenging.

The inventor has found that ticagrelor could be solubilized using a cyclodextrin. Long-term storage stability could be obtained using an aqueous solution of pH of 5.5 to 9, and importantly in the range of pH 7.0 to 8.0. The use of an organic co-solvent or surfactant were avoided, which reduced degradation risks. Particularly, the use of polyethylene glycol was avoided. The invention provides a clear ticagrelor formulation with good solubility and long-term storage stability as required for formulations in the pharmaceutical and medical field.

By the term “inclusion complex” as used herein, is meant a chemical complex in which one chemical compound, the host, has a cavity into which a guest compound can be accommodated. The interaction between the host and guest involves van der Waals bonding. Compounds suitable for providing an inclusion complex are cyclodextrins.

Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit has secondary hydroxyl groups at the 2 and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities provide a haven for hydrophobic organic compounds, which can fit all, or part of their structure into these cavities. This process, known as inclusion complexation, may result in increased apparent aqueous solubility and stability for the complexed drug; however, the degree of stabilization will vary from drug to drug. The complex is stabilized by hydrophobic interactions and does not involve the formation of any covalent bonds.

Chemical modification of the parent cyclodextrins (usually at the hydroxyl moieties) has resulted in derivatives with sometimes improved safety while retaining or improving the complexation ability of the cyclodextrin. Of the numerous derivatized cyclodextrins prepared to date, only two appear to be commercially viable; the 2-hydroxypropyl derivatives (HP-β-CD or HPβCD), neutral molecules being commercially developed by Janssen and others, and the sulfoalkyl ether derivatives (SAE-β-CD or SAE-CD), being developed by CyDex Pharmaceuticals, Inc. The SAE-CDs are a class of negatively charged cyclodextrins, which vary in the nature of the alkyl spacer, the salt form, the degree of substitution and the starting parent cyclodextrin. The sodium salt of the sulfobutyl ether derivative of beta-cyclodextrin, with an average of about 7 substituents per cyclodextrin molecule (SBE7-β-CD), is being commercialized by CyDex Pharmaceuticals, Inc. (Kansas) as CAPTISOL® cyclodextrin.

Preferably the cyclodextrin is selected from a hydroxypropyl-beta-cyclodextrin and a sulfobutylether derivative of a beta-cyclodextrin.

More preferably the cyclodextrin is hydroxypropyl-beta-cyclodextrin. The selection of the HPβCD cyclodextrin was superior to SBECD cyclodextrin. A lower amount of HPβCD was required to solubilize ticagrelor as compared to SBECD cyclodextrin. A broader range of HPβCD concentrations provided clear aqueous ticagrelor solutions as compared to SBECD. The results are provided in Example 1.

In a preferred embodiment, ticagrelor is the only active pharmaceutical ingredient present in the composition. Alternatively, an additional active ingredient can be included. Preferably the additional active ingredient is not prasugrel.

The present invention provides an aqueous liquid formulation comprising an inclusion complex of ticagrelor and an aqueous liquid carrier.

### Concentrated Ticagrelor Compositions for Bolus Injection or Short-Term Infusion

Preferably the aqueous ticagrelor composition is provided for infusion and has a volume of 15 to 40 ml; more preferably 20 to 35 ml; most preferably 25 to 30 ml. This volume range is typical for a composition according to an embodiment of the invention and use in an intravenous administration of the aqueous ticagrelor composition.

Alternatively, to an intravenous administration, the aqueous ticagrelor solution can be administered as an injection. For this purpose, the volume is preferably between 5 to 15 ml. An injection is particularly suitable for treatment of an acute situation, as the ticagrelor is made available rapidly, over a short period of time.

In a preferred embodiment the aqueous ticagrelor composition comprises 2-15 mg/ml ticagrelor; more preferably 4-14 mg/ml ticagrelor; even more preferably 6-13 mg/ml ticagrelor; most preferably 7-12 mg/ml ticagrelor or 8-10 mg/ml ticagrelor. This amount of ticagrelor is relevant for therapy by injection or short-term infusion. A selection of a lower amount requires a higher volume to be administered to a patient in need of a ticagrelor responsive medical indication. A higher amount of ticagrelor causes problems with the solubility of the active ingredient, especially in cases of an administration by injection which involves a small volume.

Most preferably the aqueous ticagrelor solution comprising 15-40% w/w, more preferably 20-35% w/w, even more preferably 22-34% w/w, most preferably 23-33% w/w hydroxypropyl-beta-cyclodextrin. The amount of cyclodextrin selected is an amount sufficient to enclose a therapeutically relevant amount of cyclodextrin and to provide a clear ticagrelor solution. This range is acceptable for medical administration.

In a preferred embodiment of the aqueous ticagrelor composition according to the invention, organic co-solvents are excluded. The use of organic co-solvents is not required to improve the solubility of ticagrelor. The avoidance of organic co-solvents provides a better compatibility of the product for an intravenous administration. The aqueous solutions of the invention are devoid of polyethylene glycol.

Preferably the aqueous ticagrelor composition has an osmolality between 350-900 mOsm/kg. This osmolality is advantageous in an intravenous administration to a patient in need of ticagrelor treatment.

In a preferred embodiment of the aqueous ticagrelor composition, the ticagrelor has a D90 particle size below 10 micrometers; more preferably the D90 particle size is below 9 micrometers; even more preferably the D90 particle size is below 8 micrometers; most preferably the D90 particle size is below 7 micrometers. Selection of the indicated particle size makes it easier to incorporate ticagrelor in cyclodextrin. The solubility of ticagrelor included in a cyclodextrin inclusion complex is improved.

A method for the measurement of particle size of an active ingredient, is well-known to a person skilled in the art of formulations. The method used in the present invention is by Malvern Mastersizer dry powder method.

The storage periods obtained for an aqueous ticagrelor solution according to an embodiment of the invention are prolonged over currently available solutions obtained by crushing Brillinta® tablets and mixing them with water. The storage stability achieved is particularly relevant for use of the ticagrelor aqueous solutions in pharmaceutical supplies and storage.

In a preferred embodiment of the aqueous ticagrelor composition, the composition has a storage stability of at least 3 months in accelerated storage conditions at 40° C. and 75% Relative Humidity (RH). More preferably said storage stability is at least 6 months; even more preferably at least 9 months; most preferably at least 12 months. A satisfactory stability of 6 months at 40° C. and 75% RH corresponds to a shelf life of 24 months at room temperature of 25° C.

“Storage stability” as used herein means that the total impurity level is below 0.5%.

Most preferably the aqueous ticagrelor composition is a solution consisting of:
5-15 mg/ml ticagrelor,
15-40% w/w of a hydroxypropyl-beta-cyclodextrin,
5 mM-20 mM of phosphate buffer,
wherein the pH is between 5.5 and 8.

The composition provided above is simple and easy to manufacture. The limited number of ingredients reduced the formation of impurities and side products.

Optionally the aqueous ticagrelor composition may including a tonicity modifier, such as sodium chloride. Preferably the osmolality of the aqueous ticagrelor composition between 350-900 mOsm/kg, more preferably between 360-800 mOsm/kg, even more preferably between 370-700 mOsm/kg, most preferably between 380 and 600 mOsm/kg.

In some embodiment, the aqueous pharmaceutical ticagrelor solution comprises a 5 w/v % dextrose solution.

In a further aspect the invention provides an aqueous ticagrelor composition for use in a ticagrelor responsive medical treatment; wherein the aqueous ticagrelor composition is as previously described.

Preferably the aqueous ticagrelor composition is provided for intravenous administration and the composition is administered as an injection or short-term infusion. This form is advantageous for unconscious patients who need immediate treatment. The short-term infusion is preferably between 5 and 30 minutes, more preferably between 10 and 25 minutes; most preferably between 15 and 20 minutes.

In alternative embodiment the aqueous ticagrelor composition is provided as a solution for nasal gastric administration or as a drink. This form is advantageous for conscious patients that have difficulties with swallowing.

Preferably the aqueous ticagrelor composition according to an embodiment of the invention is provided for use in the treatment of acute coronary syndrome (ACS), myocardial infarction (MI), ischemic stroke, transient ischemic attack (TIA) or for the reduction of platelet-tumor cell interactions in a patient in need thereof.

An aqueous composition according to an embodiment of the invention has the advantage in a medical therapy that it is faster acting than a tablet; has a higher bio availability than a tablet and provides a solution to patients who have difficulties swallowing a tablet. Especially in acute situations, an intravenous infusion or injection is advantageous. The availability of a ready-to-use aqueous solution according to an embodiment of the invention is preferred over the crushing of a tablet and addition to water and over dissolving an orodispersible tablet in water, because of time-savings and accuracy of dosing.

More preferably the aqueous ticagrelor composition according to an embodiment of the invention is for use in a monotherapy or in a dual antiplatelet therapy (DAPT). Preferably the dual antiplatelet therapy comprises acetyl salicylic acid or salts thereof as second active ingredient in addition to ticagrelor.

In a further aspect the invention provides a unit dose composition for delivery of 15-180 mg ticagrelor comprising an aqueous ticagrelor solution according to an embodiment to the invention. The dose of ticagrelor for administration to a patient may range from 15 mg to 180 mg, more preferably from 30 mg to 90 mg and most preferably from 65 to 75 mg.

Preferably the unit dose composition comprises 2000-4000 mg of cyclodextrin, more preferably 2200-3000 mg of cyclodextrin, even more preferably 2400-2800 mg of cyclodextrin, most preferably 2500 mg of cyclodextrin.

In a preferred embodiment of a unit dose composition according to the invention for bolus injection or short-time infusion, the unit dose composition comprises 5% dextrose in water (D5W) as diluent. Normal saline and Ringer's lactate solution were not suitable as diluent because they provided turbid solutions.

“Normal saline” as used herein, refers to the commonly used phrase for a solution of 0.90% w/v sodium chloride, 308 mOsm/l or 9.0 g per liter. Synonyms are physiological saline or isotonic saline.

“Ringer's lactate solution” as used herein, refers to sodium lactate solution, also called Hartmann's solution. It is a mixture of sodium chloride, sodium lactate, potassium chloride and calcium chloride in water. One liter of Ringer's lactate solution contains 130-131 mEq of sodium ion, 109-111 mEq of chloride ion, 28-29 mEq of lactate ion, 4-5 mEq of potassium ion and 2-3 mEq of calcium ion. Ringer's lactate has an osmolarity of 273 mOsm/l and a pH of 6.5.

The unit dose compositions according to an embodiment of the invention provide for treatment with ticagrelor together with a good fluid balance with minimal hypotonicity or hypertonicity. The compositions are advantageous for people who cannot take fluids orally and have developed or are in danger of developing dehydration or hypovolemia.

In a further aspect, the invention provides a method of treatment of a patient suffering from a ticagrelor-responsive disease, wherein the patient is treated with a clear and aqueous ticagrelor solution having a stability of at least one month, preferably three months, even more preferably at least six months. Preferably the clear and aqueous ticagrelor solution is a ready-to-use solution. More preferably ticagrelor is enclosed in a cyclodextrin thereby providing a ticagrelor-cyclodextrin inclusion complex. Preferably the clear and aqueous ticagrelor solution has a pH of 5.5-9; more preferably a physiological pH of 7-8. More preferably the clear and aqueous ticagrelor solution comprises a buffering agent, most preferably a phosphate buffer.

In a fourth aspect, the invention provides a method for the manufacturing of an aqueous ticagrelor composition according to an embodiment of the invention, comprising the steps of:
preparing an aqueous solution of pH 5.5-9
introducing a cyclodextrin amount for inclusion of a pre-determined amount of ticagrelor,
adding the pre-determined amount of ticagrelor, thereby obtaining an inclusion complex of ticagrelor in cyclodextrin.

The aqueous solution preferably has a pH of 5.8-8.5; more preferably 6.0-8.2; most preferably 7.0-8.0. To maintain the indicated pH range the solution is preferably comprising a buffering agent.

Preferably a heating step is applied prior to the addition of ticagrelor. This is beneficial for the reduction of the amount of cyclodextrin needed to dissolve a selected amount of ticagrelor. Ticagrelor is preferably added to a solution at a temperature of 30 to 45° C.

In a fifth aspect, the invention provides medical uses for the ticagrelor iv compositions.

The invention provides an aqueous ticagrelor solution for intravenous administration, for use in the treatment of acute coronary syndrome (ACS), myocardial infarction (MI), ischemic stroke, transient ischemic attack (TIA) or for the reduction of platelet-tumor cell interactions in a patient in need thereof.

The invention also provides an aqueous ticagrelor solution for intravenous administration, for use in a dual antiplatelet therapy (DAPT), preferably comprising acetyl salicylic acid or salts thereof as second active ingredient in addition to ticagrelor.

In a preferred embodiment, the aqueous ticagrelor iv solution has a storage stability of at least 3 months in accelerated storage conditions at 40° C. and 75% Relative Humidity.

The ingredients of the aqueous ticagrelor iv solution are preferably as previously described.

### Dilute Ticagrelor Compositions for Infusion

In a further aspect, the invention provides an aqueous pharmaceutical ticagrelor solution, comprising an inclusion complex of ticagrelor in a cyclodextrin, wherein the aqueous pharmaceutical ticagrelor solution comprises
0.10-14.0 mg/ml ticagrelor and
20-100 mg/ml of cyclodextrin in a quantity for solubilization of the ticagrelor in the selected volume of aqueous pharmaceutical solution,
   wherein the composition has a pH between 5.5-9 endpoints included, and the aqueous pharmaceutical solution has a volume of 25 to 1000 ml.

Unexpectedly it was found that ticagrelor can be solubilized in dilute concentrations in an aqueous medium. This is of interest in the pharmaceutical field for the treatment of ticagrelor-responsive conditions.

Preferably said aqueous pharmaceutical ticagrelor solution comprises 0.1-10.0 mg/ml ticagrelor, more preferably 0.2-8 mg/ml ticagrelor, even more preferably 0.3-6.0 mg/ml ticagrelor, most preferably 0.4-5.0 mg/ml ticagrelor or 0.5 mg-2.0 mg/ml. In a preferred embodiment, ticagrelor is the only active ingredient present in the aqueous pharmaceutical ticagrelor solution.

In a preferred embodiment, the osmolality is between 300-900 mOsm/kg. More preferably the osmolality is between 350-850 mOsm/kg, even more preferably 400-800 mOsm/kg, most preferably 450-750 mOsm/kg.

In a preferred embodiment the cyclodextrin is selected from a hydroxypropyl-beta-cyclodextrin and a sulfobutylether of a beta-cyclodextrin. More preferably the cyclodextrin is a hydroxypropyl-beta-cyclodextrin. Most preferably the cyclodextrin is (2-hydroxypropyl)-beta-cyclodextrin.

Preferably said aqueous pharmaceutical ticagrelor solution has a pH between 6.0-8.5 more preferably 6.5-8.0, even more preferably 6.8-7.8, most preferably around 7.5.

Preferably said aqueous pharmaceutical ticagrelor solution has a volume of 30 to 750 ml, more preferably 40 to 700 ml, even more preferably 50 to 650 ml, most preferably 100-250 ml.

In a preferred embodiment an organic co-solvent, such as polyethylene glycol, is excluded from a composition according to the invention. In a preferred embodiment a surfactant is excluded from a composition according to the invention. In a most preferred embodiment, an organic co-solvent and a surfactant are excluded from a composition according to the invention.

The dilute aqueous pharmaceutical ticagrelor solution according to an embodiment of the invention has a storage stability of at least 3 months at 25° C. and 60% Relative Humidity. More preferably the aqueous pharmaceutical ticagrelor solution has a storage stability of at least 4, 5, 6, 12, 18, or 24 months as measured at 25° C. and 60% Relative Humidity.

In some embodiment, the aqueous pharmaceutical ticagrelor solution comprises a pharmaceutically acceptable buffer. Preferably said buffer is a phosphate buffer. In some embodiments, a buffer is present in a concentration of 0,005 M to 0.1 M, preferably 0,007 M to 0,010 M, even more preferably 0.0108 M to 0,010 M, most preferably 10 mM. More preferably a 10 millimolar (mM) phosphate buffer. 1 millimolar=1 mM=1 mmol per liter.

In some embodiment, the aqueous pharmaceutical ticagrelor solution comprises a 5 w/v % dextrose solution.

In a further aspect, the aqueous pharmaceutical ticagrelor solution according to an embodiment of the invention is for use as a medicine.

In a preferred embodiment said aqueous pharmaceutical ticagrelor solution is administered as an infusion.

Preferably the aqueous pharmaceutical ticagrelor solution according to an embodiment of the invention is for use in the treatment of acute coronary syndrome (ACS), myocardial infarction (MI), ischemic stroke, transient ischemic attack (TIA) or for the reduction of platelet-tumor cell interactions in a patient in need thereof.

Preferably the aqueous pharmaceutical ticagrelor solution according to an embodiment of the invention is for use in a dual antiplatelet therapy (DAPT), preferably comprising acetyl salicylic acid or salts thereof as second active ingredient in addition to ticagrelor.

In a further aspect, the invention provides a ready-to-use infusion container comprising a composition according to an embodiment of the invention.

Preferably said composition comprises 3000-16000 mg, more preferably 4000-15000 mg, even more preferably 5000-10000 mg, most preferably 6000-8000 mg of a cyclodextrin. That cyclodextrin is preferably a hydroxypropyl-beta-cyclodextrin. Most preferably the cyclodextrin is (2-hydroxypropyl)-beta-cyclodextrin.

Preferably said composition comprises 5 w/v % dextrose or 0.9 w/v % sodium chloride. These diluents contribute to the compatibility of the composition for use in intravenous administration.

Preferably the ready-to-use infusion container is a bag or bottle.

In a preferred embodiment, ticagrelor is the only active ingredient present in the ready-to-use infusion container.

In a final aspect, the invention provides a method for the manufacturing of an aqueous pharmaceutical ticagrelor composition according to an embodiment of the invention, comprising the steps of:
preparing an aqueous solution of pH 5.5-9 preferably comprising a buffering agent, more preferably comprising a phosphate buffer,
introducing a cyclodextrin amount for inclusion of a pre-determined amount of ticagrelor,
adding the pre-determined amount of ticagrelor, thereby obtaining an inclusion complex of ticagrelor in cyclodextrin.

Preferably heating is applied prior to the addition of ticagrelor. This is beneficial for the obtaining a ticagrelor-cyclodextrin inclusion complex.

In a preferred embodiment, the ticagrelor used in a method according to an embodiment of the invention has a D90 particle size below 10 micrometers when tested using a Malvern mastersizer.

### Alternative Solubilizer for Ticagrelor

In addition, it was found that vitamin E TPGS may be used as solubilizer for ticagrelor in the preparation of aqueous ticagrelor solutions with improved storage stability.

In a final aspect, the invention provides an aqueous ticagrelor solution, comprising ticagrelor and a solubilizer for ticagrelor with a storage stability of at least 3 months, measured at 40° C. and 75% Relative Humidity or at 25° C. and 60% Relative Humidity; wherein said solubilizer is vitamin E TPGS.

Preferably said aqueous ticagrelor solution comprising vitamin E TPGS as solubilizer is for use as a medicine; preferably for parenteral administration; more preferably for intravenous administration.

Preferably said aqueous ticagrelor solution comprising vitamin E TPGS as solubilizer is free of organic co-solvents.

Preferably said aqueous ticagrelor solution comprising vitamin E TPGS as solubilizer comprises 0.6-20 mg ticagrelor per ml solution. More preferably the ticagrelor concentration is 1.0-15 mg/ml; even more preferably 5.0-10 mg/ml; most preferably 6.0-9.0 mg/ml.

Preferably said aqueous ticagrelor solution comprises 2.5-10.0 w/v % vitamin E TPGS.

Preferably said aqueous ticagelor solution comprises 2.5 w/v vitamin E TPGS in water and 10 mg ticagrelor per ml solution.

Preferably said aqueous ticagelor solution comprises 5.0 w/v vitamin E TPGS in water and 10-12 mg ticagrelor per ml solution.

Preferably said aqueous ticagelor solution comprises 10.0 w/v vitamin E TPGS in water and 10-15 mg ticagrelor per ml solution.

The invention is further illustrated by way of examples. The examples are non-exhaustive.

### EXAMPLES

### Example 1

In a first example two different types of cyclodextrin were used and compared for the solubilization of ticagrelor. An unbuffered stock solution of HPβCD or SBECD was made in water at the target concentrations of 20 w/w %, 25 w/w %, 30 w/w %, 35 w/w % and 40 w/w %. Ticagrelor was added slowly under vortex. Ticagrelor was used at concentrations of 5, 10 or 14 mg/ml in milli Q water. The ticagrelor-cyclodextrin solutions were left on a shaking platform. No sonification or heat was applied.

From the results in Table 1 and 2 it follows that HPβCD was able to dissolve ticagrelor in a broader range of cyclodextrin and ticagrelor concentrations tested. Clear aqueous solutions with 5 mg/ml ticagrelor in HPβCD were obtained with 25 w/w %, 30 w/w %, 35 w/w % and 40 w/w % HPβCD.

TABLE 1
Solubility of ticagrelor in HPβCD

| | | Appearance of Ticagrelor in cyclodextrin | | | | | |
|---|---|---|---|---|---|---|---|
| HPβCD | | | After | After | | | |
| conc. in | Ticagrelor | After 5 | 30 | 60 | After | After | |
| MilliQ | conc. | min of | min of | min of | 3 h of | overnight | Flocculation |
| (% w/w) | (mg/ml) | shaking | shaking | shaking | shaking | shaking | test |
| 40% | 5 | + | + | + | + | + | passed |
| | 10 | − | +/− | +/− | +/− | + | passed |
| | 14 | − | +/− | +/− | +/− | +/−* | n.a. |
| 35% | 5 | +/− | +/− | +/− | +/− | + | passed |
| | 10 | − | +/− | +/− | +/− | + | did not pass |
| | 14 | − | +/− | +/− | +/− | +/− | n.a. |
| 30% | 5 | − | +/− | +/− | +/− | + | passed |
| | 10 | − | − | − | − | − | n.a. |
| | 14 | − | − | − | − | − | n.a. |
| 25% | 5 | − | +/− | +/− | +/− | +* | did not pass |
| | 10 | − | − | − | − | − | n.a. |
| | 14 | − | − | − | − | − | n.a. |
| 20% | 5 | − | +/− | +/− | +/− | − | n.a. |
| | 10 | − | − | − | − | − | n.a. |
| | 14 | − | − | − | − | − | n.a. |

+ clear, appeared completely dissolved
− translucent solution with precipitation
+/− clear solution with precipitation
*after one hour of sonication

TABLE 2
Solubility of ticagrelor in SBECD

| | | Appearance of Ticagrelor in cyclodextrin | | | | | |
|---|---|---|---|---|---|---|---|
| SBECD | | After | After | After | | | |
| conc. in | Ticagrelor | 5 min | 30 min | 60 min | After | After | |
| MilliQ | conc. | of | of | of | 3 h of | overnight | Flocculation |
| (% w/w) | (mg/ml) | shaking | shaking | shaking | shaking | shaking | test |
| 40% | 5 | − | +/− | +/− | +/− | + | passed |
| | 10 | − | − | − | − | − | n.a. |
| | 14 | − | − | − | − | − | n.a. |
| 35% | 5 | − | +/− | +/− | +/− | − | n.a. |
| | 10 | − | − | − | − | − | n.a. |
| | 14 | − | − | − | − | − | n.a. |
| 30% | 5 | − | − | − | − | − | n.a. |
| | 10 | − | − | − | − | − | n.a. |
| | 14 | − | − | − | − | − | n.a. |
| 25% | 5 | − | − | − | − | − | n.a. |
| | 10 | − | − | − | − | − | n.a. |
| | 14 | − | − | − | − | − | n.a. |
| 20% | 5 | − | − | − | − | − | n.a. |
| | 10 | − | − | − | − | − | n.a. |
| | 14 | − | − | − | − | − | n.a. |

+ clear, appeared completely dissolved
− translucent solution with precipitation
+/− clear solution with precipitation

As a conclusion Ticagrelor could be dissolved by leaving it on a shaking platform. No sonication was applied. HPβCD can be used at Ticagrelor concentrations of 5 mg/ml, using cyclodextrin at 40% w/w, 35% w/w or 30% w/w in milliQ water. These solutions remained clear at least for the three days testing at room temperature and several days at 4° C.

### Example 2

After the experiments depicted in Example 1, further optimization was carried out with the selection of a suitable pH range to ensure long-term stability of the aqueous ticagrelor-cyclodextrin inclusion complex.

The following composition as provided in Table 3 was prepared.

TABLE 3
Composition for storage stability testing.

| | mg/ml |
|---|---|
| Ticagrelor | 6 |
| HPβCD 40% w/w | 452 |
| Acetate or Phosphate Buffer pH 4.5 to 6.5 | Q.S to 1 ml |

HPβCD was dissolved in a buffer solution of pH 4.5, 5.5 or 6.5 prepared separately in water. Once a clear solution was obtained, ticagrelor was dissolved in the buffer solution under constant stirring. The ticagrelor in buffer solution was filtered through a 0.22 micron filtered and filled in USP Type I glass vials. The vials were stoppered and stored. All precautions were taken during manufacturing, such as N2 purging and avoiding direct exposure to light. The vials were stored at 40° C. and 75% Relative Humidity (RH).

To determine the stability of the formulations, batches were evaluated using a related substance method on HPLC. The data of these batches is enumerated below in Table 4.

A Gradient HPLC method was used to analyze impurities in formulations using a YMC-Pack Pro C18 column (100×4.6 mm, S-3 μm 12 nm). Good separation was obtained for all the impurities.
Amine impurity: (1S,2S,3R,5S)-3-(7-amino-5-(propylsulfanyl)-3H-[1,2,3]triazolo[4,5-d]pyrimidin-3-yl)-5-(2-hydroxyethoxy)cyclopentane-1,2-diol. This is a process related degradant impurity.
Regiomer impurity: (1S, 2S, 3R, 5S)-3-((3-((1R,2S)-2-(3,4-Difluorophenyl)cyclopropyl)-5-(propylsulfanyl)-3H-[1,2,3]triazolo[4,5-d]pyrimidin-7-yl)amino)-5-(2-hydroxyethoxy)cyclopentane-1,2-diol. This is a process related degradant impurity.
Acetal impurity: 2-[[(3aR,4S,6S,6aS)-6-[7-[1R,2S)-2-(3,4-difluorophenyl)-cyclopropyl]amino]-5-(propylsulfanyl)-3H-[1,2,3]triazolo-[4,5-d]pyrimidin-3-yl]-2,2-dimethyltetrahydro-2H-3aHcyclopenta[d][1,3]dioxol-4-yl]oxy]ethan-1-ol.

This is a process related impurity.
Triol impurity: (1S,2R,3S,4R)-4-(7-((1R,2S)-2-(3,4-difluorophenyl)cyclopropylamino)-5-(propylthio)-3H-[1,2,3]triazolo[4,5-d]pyrimidin-3-yl)cyclopentane-1,2,3-triol.

This is a process related impurity.

It was observed that only regiomer impurity increased in 4 weeks 40° C. and 75% RH at almost 0.3% level; specification limit 0.3%. Hence to optimize the stability of the product further, investigations were carried out at pH 7 to 8.

### Example 3

Following the experiment described in example 2, a storage stability study at pH 7.5 was conducted

First HPβCD was dissolved in a phosphate buffer solution of pH 7.5 prepared separately in water. Once a clear solution was obtained ticagrelor was dissolved in the solution under constant stirring. The solution was filtered through a 0.22 micrometer filter and filled in USP Type I amber colored glass vials. The vials were stoppered and stored. All precautions were taken during manufacturing, such as N2 purging and avoiding direct exposure to light.

TABLE 4
Stability test of ticagrelor-cyclodextrin inclusion complex in aqueous solution at pH 4.5, 5.5 and 6.5 after storage at 40° C. and 75% RH

| | | Timepoint | | | | | | | | |
|---|---|---|---|---|---|---|---|---|---|---|
| | | T = 0 | | | T = 2 weeks | | | T = 4 weeks | | |
| | | Condition | | | | | | | | |
| | | pH 4.5 | pH 5.5 | pH 6.5 | pH 4.5 | pH 5.5 | pH 6.5 | pH 4.5 | pH 5.5 | pH 6.5 |
| Relative | | Average | Average | Average | Average | Average | Average | Average | Average | Average |
| retention | | impurity | impurity | impurity | impurity | impurity | impurity | impurity | impurity | impurity |
| time | | content | content | content | content | content | content | content | content | content |
| (minutes) | Impurity | (%) | (%) | (%) | (%) | (%) | (%) | (%) | (%) | (%) |
| 0.45 | Amine impurity | 0.05 | 0.04 | 0.05 | 0.05 | 0.05 | 0.05 | 0.05 | 0.04 | 0.05 |
| 0.70 | Oxidative impurity 1 | 0.00 | 0.00 | 0.00 | 0.00 | 0.00 | 0.00 | 0.01 | 0.01 | 0.00 |
| 0.70 | Oxidative impurity 2 | 0.00 | 0.00 | 0.00 | 0.00 | 0.00 | 0.00 | 0.02 | 0.02 | 0.04 |
| 0.97 | Triol impurity | 0.05 | 0.05 | 0.06 | 0.05 | 0.05 | 0.06 | 0.05 | 0.05 | 0.05 |
| 1.02 | Regiomer impurity | 0.00 | 0.00 | 0.00 | 0.00 | 0.00 | 0.00 | 0.42 | 0.30 | 0.35 |
| 1.50 | Acetal impurity | 0.05 | 0.05 | 0.06 | 0.06 | 0.05 | 0.06 | 0.05 | 0.05 | 0.06 |
| 1.52 | | 0.03 | 0.02 | 0.03 | 0.04 | 0.03 | 0.04 | 0.03 | 0.02 | 0.03 |
| Total Impurities | | 0.36 | 0.33 | 0.36 | 0.40 | 0.36 | 0.41 | 0.80 | 0.65 | 0.72 |
| (%) | | | | | | | | | | |
| Sum | | 0.22 | 0.16 | 0.22 | 0.23 | 0.21 | 0.23 | 0.63 | 0.45 | 0.56 |
| Impurities >0.05 | | | | | | | | | | |
| (%) | | | | | | | | | | |

TABLE 5
Composition for storage stability testing.

| | mg/ml |
|---|---|
| Ticagrelor | 6 |
| HPβCD 40% w/w | 452 |
| Phosphate buffer pH 7.5 | Q.S to 1 ml |

TABLE 6
Storage stability study of ticagrelor-cyclodextrin inclusion complex in
aqueous solution at pH 7.5 stored at 40° C. and 75% Relative Humidity.

| | | Time point 40° C. and 75% RH | | | | |
|---|---|---|---|---|---|---|
| | | | T = 4 | T = 8 | T = 12 | T = 24 |
| | | T = 0 | weeks | weeks | weeks | Weeks |
| Relative | | Assay ticagrelor (%) | | | | |
| retention | | 104.27 | 104.47 | 102.59 | 102.31 | 104.39 |
| time | | impurity | impurity | impurity | impurity | impurity |
| (minutes) | Impurity ID | (%): | (%): | (%): | (%): | (%): |
| 0.45 | Amine impurity | 0.04 | 0.05 | 0.05 | 0.05 | 0.07 |
| 0.97 | Triol impurity | 0.04 | 0.05 | 0.05 | 0.04 | 0.05 |
| 1.03 | Regiomer impurity | 0.00 | 0.01 | 0.04 | 0.08 | 0.16 |
| 1.49 | Acetal impurity | 0.05 | 0.05 | 0.05 | 0.05 | 0.05 |

| | | | | | |
|---|---|---|---|---|---|
| Total Impurities (%) | 0.31 | 0.32 | 0.29 | 0.41 | 0.35 |
| Sum Impurities >0.05 (%) | 0.11 | 0.14 | 0.15 | 0.23 | 0.33 |

Based on the results of the stability study, as summarized in Table 6, it was concluded that good storage stability was obtained at accelerated storage conditions of 40° C. and 75% Relative Humidity. The regiomer impurity was well under control and no other impurity was of a concern.

### Example 4

In a further experiment, to optimize the concentration of HPβCD below 40% w/w, heat at 40° C. was applied at concentrations where a clear solution was difficult to obtain to help dissolve the target ticagrelor dose.

Direct physical stability data as obtained from the ticagrelor 5 mg/ml concentrate and with the flocculation test (20 μl sample material in 1 ml diluent) are shown in Table 7. Table 7 contains data on assay, purity, osmolality and pH.

TABLE 7
Physical stability of ticagrelor 5 mg/ml batches with varying HPβCD
concentrations. Data are sorted by HPβCD strength. Ticagrelor
concentration when diluted into dextrose or saline: 0.1 mg/ml.

| HPβCD strength | | Appearance in | Appearance |
|---|---|---|---|
| (% w/w) | Appearance, undiluted | 5% dextrose | in NaCl 0.9% |
| 32.5 | Clear solution | Clear solution | Clear solution |
| 30.0 | Clear solution | Clear solution | Turbid |
| 27.5 | Turbid; clear after heating to 40° C. | Clear solution | Turbid |
| 25.0 | Turbid; clear after heating to 40° C. | Clear solution | Turbid |
| 22.5 | Turbid; clear after heating to 40° C. | Clear solution | Turbid |

Because of the poor physical stability results when diluted in saline, 32.5% w/w HPβCD was chosen for a 5 mg/ml ticagrelor formulation. Undiluted concentrate remained stable even in the refrigerator with a HPβCD concentration as low as 22.5% w/w. Such a concentration yielded a nearly isotonic formulation.

In conclusion, it was possible to dissolve 5-15 mg/ml ticagrelor together with HPβCD in the concentration range of 20-40% w/w, without the use of heat. It was possible to achieve good solubility with lower concentrations of HPβCD such as 15-20% w/w with application of heat to achieve a clear solution.

At least 15% w/w HPβCD was required to provide a clear, storage stable ticagrelor solution with a concentration that is relevant for injection or intravenous administration.

### Example 5

From the results obtained in Example 4 it follows that the concentration of excipients may be such that the resulting ticagrelor solution is hypertonic. The osmolality and pH of several batches was checked. The solutions had a 19 mM phosphate buffer and pH 7.5. The results are provided in Table 8.

TABLE 8
pH and osmolality determination in undiluted batches.

| HPβCD | | Total | | Osmolality |
|---|---|---|---|---|
| strength | Assay | impurities | pH | undiluted |
| (% w/w) | (%) | (%) | undiluted | (mOsm/kg) |
| 32.5 | 99.26 | 0.41 | 7.68 | 813 |
| 30.0 | 107.64 | 0.44 | 7.74 | 638 |
| 27.5 | 104.27 | 0.42 | 7.69 | 549 |
| 25.0 | 103.23 | 0.41 | 7.69 | 493 |
| 22.5 | 98.15 | 0.39 | 7.67 | 392 |

Dilution studies were conducted to search for suitable diluents.

5 mg/ml ticagrelor-cyclodextrin solutions with varying amounts of HPβCD were diluted with normal saline, 5% dextrose solution or Ringer's lactate solution. The stability was screened. The ticagrelor concentration when diluted into dextrose or saline: 0.1 mg/ml The results are summarized in Table 9.

TABLE 9
Diluent tests

| HPβCD strength | | Appearance in | Appearance |
|---|---|---|---|
| (% w/w) | Appearance, undiluted | 5% dextrose | in NaCl 0.9% |
| 32.5 | Clear solution | Clear solution | Clear solution |
| 30.0 | Clear solution | Clear solution | Turbid |
| 27.5 | Turbid; clear after heating to 40° C. | Clear solution | Turbid |
| 25.0 | Turbid; clear after heating to 40° C. | Clear solution | Turbid |
| 22.5 | Turbid; clear after heating to 40° C. | Clear solution | Turbid |

In addition, a screening was conducted on the impact of buffer strength on pH and osmolality. The results are summarized in Table 10.

TABLE 10
Impact of buffer strength on pH, osmolality, assay, and impurities.

| Phosphate | | | Osmo- | Osmo- | | |
|---|---|---|---|---|---|---|
| buffer | | | lality | lality | | Total |
| pH 7.5 | | | sample | placebo | | impuri- |
| strength | pH | pH | (mOsm/ | (mOsm/ | Assay | ties |
| (mM) | sample | placebo | kg) | kg) | (%) | (%) |
| 19 | 7.70 | 7.71 | 758 | 790 | 102.58 | 0.42 |
| 10 | 7.69 | 7.72 | 724 | 692 | 102.98 | 0.42 |
| 5 | 7.66 | 7.73 | 703 | 725 | 100.49 | 0.40 |
| 0.19 | 7.18 | 7.45 | 809 | 771 | 102.23 | 0.42 |

It was concluded that a phosphate buffer of pH 7.5 at different buffer strengths had little effect on the osmolality. Except at 0.19 mM buffer strength. This buffer strength was too weak, which lead to a change in pH.

### Example 6

In a further example the impact of particle size on solubility was tested.

Two different particle size diameters for the ticagrelor active ingredient were screened, 5.5 and 15 micrometers. The pH and osmolality were not affected. Smaller particles showed a faster dissolution time, as summarized in Table 11.

The micronized ticagrelor showed significant improvement on the dissolution time. Consequently, a micronized ticagrelor with D90 of less than 10 micrometers is preferred.

With the term “D90” as used herein, is meant that at least 90% of the particles present have a size that is less than the target particle size. However, it is understood that variations in input particle size distribution (PSD) of ticagrelor would be possible and it will have an impact on the dissolution rate of ticagrelor.

TABLE 11
Impact of particle size

| Particle distribution, | Dissolution | | Osmolality |
|---|---|---|---|
| D90 (0.9) | time | pH | (mOsm/kg) |
| 5.5 μm, micronized | 33 min | 7.68 | 751 |
| 15 μm, unmicronized | 125 min | 7.70 | 753 |

### Example 7

To optimize the HPβCD concentration and the pH of the solution for intravenous use, a 12 week/3 months stability study was conducted. A composition of 32.5% w/w HPβCD with 5 mg/ml ticagrelor at pH 7 to 8 was prepared and stored. Its stability was tested at regular intervals.

A comparison of stability profile at 3 different pH—7, 7.5 and 8—was carried out as below, the manufacturing process for all 3 formulations was kept constant with buffer strength at 19 mM. The results are summarized in Tables 12 to 14

TABLE 12
Storage stability in amber glass vials - pH 7

| | | Amber colour USP Type I | | | |
|---|---|---|---|---|---|
| | | 19 mM pH 7 Phosphate Buffer | | | |
| HPβCD | 40% w/w | T0 | 1 M | 2 M | 3 M |
| | Description | clear | clear | clear | clear |
| | Assay | 98.95 | 99.73 | 99.81 | 98.51 |
| | pH | 7.33 | 7.34 | 7.34 | 7.30 |
| RRT | Impurity | % | % | % | % |
| 0.45 | Amine | 0.04 | 0.04 | 0.04 | 0.06 |
| | impurity | | | | |
| 0.97 | Triol impurity | 0.05 | 0.02 | 0.04 | 0.04 |
| 1.03 | Regiomer | 0.00 | 0.03 | 0.06 | 0.11 |
| 1.49 | Acetal | 0.05 | 0.05 | 0.05 | 0.05 |
| | impurity | | | | |
| | Total | 0.41 | 0.27 | 0.34 | 0.40 |

| | | | | |
|---|---|---|---|---|
| Sum Impurities >0.05 (%) | 0.22 | 0.05 | 0.11 | 0.22 |

TABLE 13
Storage stability in amber glass vials - pH 7.5

| | | Amber colour USP Type I | | | |
|---|---|---|---|---|---|
| | | 19 mM pH 7.5 Phosphate Buffer | | | |
| HPβCD | 40% w/w | T0 | 1 M | 2 M | 3 M |
| | Description | clear | clear | clear | clear |
| | Assay | 104.27 | 104.47 | 102.59 | 102.31 |
| | pH | 7.40 | 7.50 | 7.48 | 7.55 |
| RRT | Impurity | % | % | % | % |
| 0.45 | Amine | 0.04 | 0.05 | 0.05 | 0.05 |
| | impurity | | | | |
| 0.97 | Triol impurity | 0.04 | 0.05 | 0.05 | 0.04 |
| 1.03 | Regiomer | 0.00 | 0.01 | 0.04 | 0.08 |
| 1.49 | Acetal | 0.05 | 0.05 | 0.05 | 0.05 |
| | impurity | | | | |
| | Total | 0.31 | 0.32 | 0.29 | 0.41 |

| | | | | |
|---|---|---|---|---|
| Sum Impurities >0.05 (%) | 0.11 | 0.14 | 0.15 | 0.23 |

TABLE 14
Storage stability in amber glass vials - pH 8.0

| | | Amber colour USP Type I | | | |
|---|---|---|---|---|---|
| | | 19 mM pH 8 Phosphate buffer | | | |
| HPβCD | 40% w/w | T0 | 1 M | 2 M | 3 M |
| | Description | clear | clear | clear | clear |
| | Assay | 103.25 | 105.88 | 105.46 | 104.46 |
| | pH | 8.07 | 8.10 | 8.04 | 8.03 |
| RRT | Impurity | % | % | % | % |
| 0.45 | Amine | 0.04 | 0.05 | 0.05 | 0.07 |
| | impurity | | | | |
| 0.97 | Triol impurity | 0.06 | 0.03 | 0.05 | 0.05 |
| 1.03 | Regiomer | 0.00 | 0.01 | 0.03 | 0.06 |
| 1.49 | Acetal | 0.05 | 0.05 | 0.05 | 0.05 |
| | impurity | | | | |
| | Total | 0.43 | 0.26 | 0.32 | 0.37 |

| | | | | |
|---|---|---|---|---|
| Sum Impurities >0.05 (%) | 0.25 | 0.09 | 0.15 | 0.23 |

From the above data it was concluded that the ticagrelor solution in HPβCD was stable in the PH range of 7 to 8.

### Example 8

To study the potential impact of the packaging material on the stability of the ticagrelor-cyclodextrin inclusion complex a composition with 32.5% w/w HPβCD was prepared with procedures and precaution's similar to previous trials, samples were stored in transparent clear glass vials and amber colored glass vials at a temperature of 40° C./75% RH. The results are shown in Table 15 and Table 16.

The results of the accelerated storage stability test indicated that after 3 months, no significant difference was observed between the two. All the samples remained clear aqueous solutions. The pH of the samples remained stable. Impurities did not change significantly.

It seems that both clear and amber colored glass vials can be used.

Compared to the results of the accelerated storage stability test on ticagrelor solutions, without the use of cyclodextrin, it is clear that the use of cyclodextrin is important to achieve a good stability. Without the cyclodextrin, 6 to 8 different impurities developed on storage. These impurities were not seen in the selected composition.

TABLE 15
Study of the potential impact of packaging.
Stability in amber color USP Type I glass.

| | | Amber colour USP Type I Glass vials | | | |
|---|---|---|---|---|---|
| | | 5 mM pH 7.5 Phosphate Buffer | | | |
| HPβCD | 32.5% w/w | T0 | 1 M | 2 M | 3 M |
| | Description | clear | clear | clear | clear |
| | Assay | 98.34 | 100.24 | 99.89 | 98.93 |
| | pH | 7.66 | 7.70 | 7.69 | 7.66 |
| RRT | Impurity | % | % | % | % |
| 0.45 | Amine impurity | 0.04 | 0.04 | 0.04 | 0.06 |
| 0.97 | Triol impurity | 0.04 | 0.02 | 0.04 | 0.05 |
| 1.03 | Regiomer | 0.00 | 0.01 | 0.02 | 0.04 |
| 1.49 | Acetal impurity | 0.05 | 0.05 | 0.04 | 0.05 |
| | Total | 0.40 | 0.22 | 0.28 | 0.33 |

| | | | | |
|---|---|---|---|---|
| Sum Impurities >0.05 (%) | 0.18 | 0.05 | 0.00 | 0.16 |

TABLE 16
Study of the potential impact of packaging.
Stability clear glass vials USP Type I.

| | | Clear USP Type I Glass vials | | | |
|---|---|---|---|---|---|
| | | 5 mM pH 7.5 Phosphate buffer | | | |
| HPβCD | 32.5% w/w | T0 | 1 M | 2 M | 3 M |
| | Description | clear | clear | clear | clear |
| | Assay | 98.34 | 101.71 | 99.69 | 98.25 |
| | pH | 7.65 | 7.65 | 7.63 | 7.58 |
| RRT | Impurity | % | % | % | % |
| 0.45 | Amine impurity | 0.04 | 0.04 | 0.04 | 0.06 |
| 0.97 | Triol impurity | 0.05 | 0.03 | 0.05 | 0.05 |
| 1.03 | Regiomer | 0.00 | 0.01 | 0.02 | 0.04 |
| 1.49 | Acetal impurity | 0.05 | 0.04 | 0.04 | 0.05 |
| | Total | 0.40 | 0.22 | 0.28 | 0.33 |

| | | | | |
|---|---|---|---|---|
| Sum Impurities >0.05 (%) | 0.22 | 0.00 | 0.05 | 0.16 |

Surprisingly it could be concluded that ticagrelor solutions can be stabilized with HPβCD in both amber colored and clear glass vials.

### Example 9

Further embodiments of the invention are provided as summarized in Table 17. Further improvement in achieving higher solubility of ticagrelor was tried with different concentrations, such as with 40% w/w HPβCD, a ticagrelor solubility of 13 mg/ml was also possible.

TABLE 17
clear aqueous solutions with ticagrelor-cyclodextrin
inclusion complex considering 65 mg dose.

| | | | Final | | | | Flocculation | Flocculation |
|---|---|---|---|---|---|---|---|---|
| | HPβCD | Ticagrelor | Vol | HPβCD | HPβCD | | in saline, | in dextrose, |
| Nr | (% w/w) | (mg/ml) | ml | mg/ml | g/vial | Appearance | 1:2 | 1:2 |
| A | 32.5 | 7.5 | 10 | 367.25 | 3.67 | Clear, | Clear | Clear |
| | | | | | | colorless | | |
| B | 32.5 | 5 | 15 | 367.25 | 5.50 | Clear, | Clear | Clear |
| | | | | | | colorless | | |
| C | 22.5 | 5 | 15 | 254.25 | 3.81 | Clear, | Clear | Clear |
| | | | | | | colorless | | |
| C | 40 | 13 | 5 | 452 | 3.39 | Clear, | Clear | Clear |
| | | | | | | colorless | | |
| D | 33 | 8.25 | 9.2 | 372.3 | 3.43 | Clear, | Clear | Clear |
| | | | | | | colorless | | |
| E | 30 | 7.5 | 10 | 339 | 3.39 | Clear, | Clear | Clear |
| | | | | | | colorless | | |

Density 1.130 gm/cc

Based on the investigations it was observed that a concentration of 5-13 mg/ml ticagrelor solution could be achieved using 20-40% w/w HPβCD. The volume of the fill content can be changed based on the dose required.

Surprisingly it was found that the target dose of 5-15 mg/ml ticagrelor contained in a small volume could be achieved by adjusting the HPβCD % and total available volume of the formulation ready to inject. Being able to contain the ticagrelor dose in a volume of 5-15 ml is highly relevant as it is a typical bolus injection volume.

### Example 10

In a further example, the maximum solubility of ticagrelor in an HPβCD solution, without the use of heat, was investigated. The results are summarized in Table 18.

Depending on the amount of ticagrelor to be delivered to a patient and the restriction of the sample volume as determined by an administration by injection or infusion, it follows that to dissolve 65-75 mg ticagrelor an amount of 2000-4000 mg of HPβCD per vial of 10 ml may be required.

TABLE 18
Concentration of HPBCD, dose and volume of formulations

| HPβCD | HPβCD | mg/ml | Ticagrelor | 75 mg | HPβCD | 65 mg | | HPβCD |
|---|---|---|---|---|---|---|---|---|
| % w/W | % w/v | HPβCD | mg/ml | dose | mg/vial | dose | | mg/vial |
| 17 | 19.21 | 192.10 | 4 | 18.75 ml | 3600 | 16.25 | ml | 3121 |
| 22.5 | 25.42 | 254.20 | 5 | 15.00 ml | 3813 | 13 | ml | 3304 |
| 30 | 33.90 | 339.00 | 8 | 9.37 ml | 3176 | 8.12 | ml | 2752 |
| 33 | 37.29 | 372.90 | 9 | 8.33 ml | 3107 | 7.22 | ml | 2692 |
| 40 | 45.2 | 452.00 | 13 | 7.76 ml | 3507 | 5 | ml | 2260 |

Density of the HPβCD solution 1.130 gm/cc

Surprisingly the solutions provided in Table 18 were compatible with diluents to provide infusions, specifically with dextrose 5% in water.

### Example 11

In another embodiment of this invention, a highly stable clear solution of ticagrelor could be obtained by applying appropriate heat to the solution during preparation thus providing a completely clear solution of the formulation at desired HPβCD and ticagrelor concentrations.

To investigate the impact of temperature and hold time, a new composition was prepared as per below Table 19.

TABLE 19
Composition for temperature impact assessment.

| | mg/ml |
|---|---|
| Ticagrelor | 8 |
| HPβCD 30% w/w | 329 |
| Phosphate Buffer pH 7.5 in water | Q.S to 1 ml |

In first step a phosphate buffer at pH 7.5 was prepared and the buffered solution was heated to 40° C.-45° C. HPβCD was added to the buffered solution under continuous mixing. Once a clear solution was obtained, ticagrelor was dispersed into the HPβCD solution and mixed until a clear solution was obtained. It usually took 30 mins to 4 hours depending on batch size.

Then this solution was filtered through a 0.22-micron filter and packed in suitable clear or amber colored glass vials

TABLE 20
Bulk hold study at 45° C.

Time points @ 45° C.-
Bulk hold in Glass vials

| | |
|---|---|
| T = 1 h | T = 110 h |

Assay Ticagrelor (%)

| | | |
|---|---|---|
| Relative | 107.87 | 109.10 |

| | | |
|---|---|---|
| retention | | Average content |
| time: | Impurity | impurity (%): |

| | | | |
|---|---|---|---|
| 0.45 | Amine impurity | 0.05 | 0.07 |
| 0.97 | Triol impurity | 0.04 | 0.05 |
| 1.33 | | 0.07 | 0.03 |
| 1.47 | Acetal impurity | 0.04 | 0.04 |

| | | |
|---|---|---|
| Total Impurities (%) | 0.20 | 0.28 |
| Sum Impurities >0.05 (%) | 0.12 | 0.11 |

TABLE 21
Bulk Hold study at 25° C. and 40° C.

| | Bulk solution @ sealed vials | | |
|---|---|---|---|
| | Ticagrelor 8 mg/ml in 30% w/w HPβCD, | | |
| | phosphate buffer pH 7.5, 25° C. and 40° C. | | |
| | | Time point | |
| | | T = 1 M, 25° C. | T = 1 M, 40° C. |
| Relative | | Assay Ticagrelor (%) | |
| retention | | 107.22 | 105.16 |
| time: | Impurity | Average content impurity (%): | |
| 0.13 | Amine | 0.06 | 0.06 |
| | impurity | | |
| 0.96 | Triol impurity | 0.01 | 0.00 |
| 1.03 | | 0.02 | 0.02 |
| 1.07 | Regiomer | 0.03 | 0.08 |
| | impurity | | |
| 1.33 | | 0.03 | 0.08 |
| 1.78 | Acetal | 0.03 | 0.00 |
| | impurity | | |

| | | |
|---|---|---|
| Total Impurities (%) | 0.23 | 0.28 |
| Sum Impurities >0.05 (%) | 0.06 | 0.21 |

TABLE 22
Bulk hold at 30° C.

Time point

| | |
|---|---|
| T0 | T4 W |

Assay Ticagrelor (%)

| | | |
|---|---|---|
| Relative | 105.60 | 106.14 |

| | | |
|---|---|---|
| retention | | Average content |
| time: | Impurity | impurity (%): |

| | | | |
|---|---|---|---|
| 0.45 | Amine impurity | 0.06 | 0.06 |
| 0.98 | Triol impurity | 0.05 | 0.04 |
| 1.06 | Regiomer impurity | 0.00 | 0.00 |
| 1.50 | Acetal impurity | 0.04 | 0.04 |

| | | |
|---|---|---|
| Total Impurities (%) | 0.21 | 0.21 |
| Sum Impurities >0.05 (%) | 0.10 | 0.06 |

The hold time study at temperatures between 25° C.-45° C. indicated how even 30% w/w HPβCD was capable of stabilizing ticagrelor, even after heating the solution for a prolonged period of time or keeping the bulk at an elevated temperature.

### Example 12: Aqueous Ticagrelor Solution for Oral Administration

In the present example an aqueous ticagrelor formulation is presented that is provided for oral administration. The composition is provided in Table 23.

TABLE 23
Composition of an aqueous ticagrelor
formulation for oral administration

| | | Unit Composition | |
|---|---|---|---|
| Name of Ingredient | Function | w/v (mg/ml) | w/v (%) |
| Ticagrelor | Active Ingredient | 5 | 0.5 |
| Propylene Glycol | Solvent | 114.08 | 11.41 |
| Methylparaben | Preservative | 1.80 | 0.18 |
| Propylparaben | Preservative | 0.20 | 0.02 |
| Hydroxy propyl beta | Solubilizer | 300.0 | 30.0 |
| cyclodextrin | | | |
| Sucralose | Sweetener | 4.72 | 0.472 |
| Sodium Benzoate | Preservative | 0.90 | 0.09 |
| Natural Peppermint Extract | Flavor | 0.78 | 0.08 |
| Natural Orange Flavor | Flavor | 1.10 | 0.11 |
| NaOH | pH adjustment | Q.S | Q.S |
| Purified Water q.s | Vehicle | To 100 ml | To 100 ml |

The process of manufacturing such oral solutions is straight forward. Preservatives are dissolved in propylene glycol. HPβCD was dissolved in water and ticagrelor is added into it under continuous stirring and then mixed with solution of propylene glycol. The remaining items were added in the solution and pH is adjusted to 7-8.

### Ready-to-Use Formulations

### Example 13

With the aim to formulate a ready-to-use ticagrelor aqueous composition, several diluted ticagrelor compositions were made and tested for solubility and stability.

TABLE 24
ready to use ticagrelor compositions in water

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml TCG | 0.325 mg/ml TCG | 0.10 mg/ml TCG |
| | concentration | concentration | concentration |
| | Composition 13.1 | Composition 13.2 | Composition 13.3 |
| Ticagrelor | 65 | 65 | 65 |
| Hydroxy | 8000 | 16000 | 16000 |
| Propyl beta | | | |
| cyclodextrin | | | |
| (HPβCD) | | | |
| Water | 100 ml | 200 ml | 650 ml |
| pH | 7.33 | 7.40 | 7.57 |
| Osmolarity | 96 | 90 | 22 |
| (mOsmol/kg) | | | |
| Clarity | Clear solution | Clear solution | Clear solution |

TABLE 25
ready to use ticagrelor compositions in aqueous phosphate buffer

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml TCG | 0.325 mg/ml TCG | 0.10 mg/ml TCG |
| | concentration | concentration | concentration |
| | Composition 13.4 | Composition 13.5 | Composition 13.6 |
| Ticagrelor | 65 | 65 | 65 |
| Hydroxy | 8000 | 16000 | 16000 |
| Propyl beta | | | |
| cyclodextrin | | | |
| (HPβCD) | | | |
| Phosphate | 100 ml | 200 ml | 650 ml |
| buffer pH | | | |
| 7.5 (10 mM | | | |
| buffer) | | | |
| pH | 7.58 | 7.45 | 7.6 |
| Osmolarity | 105 | 220 | 280 |
| (mOsmol/kg) | | | |
| Clarity | Clear solution | Clear solution | Clear solution |

TABLE 26
ready to use ticagrelor compositions in diluted saline water

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml TCG | 0.325 mg/ml TCG | 0.10 mg/ml TCG |
| | concentration | concentration | concentration |
| | Composition 13.7 | Composition 13.8 | Composition 13.9 |
| Ticagrelor | 65 | 65 | 65 |
| Hydroxy | 8000 | 16000 | 16000 |
| Propyl beta | | | |
| cyclodextrin | | | |
| (HPβCD) | | | |
| Normal | 100 ml | 200 ml | 650 ml |
| Saline | | | |
| (0.9 w/v % | | | |
| Nacl in | | | |
| water) | | | |
| pH | 6.57 | 6.6 | 6.90 |
| Osmolarity | 518 | 415 | 314 |
| (mOsmol/kg) | | | |
| Clarity | Clear solution | Clear solution | Clear solution |

TABLE 27
ready to use ticagrelor compositions in dextrose solution

| | RTU Infusion | RTU Infusion | RTU Infusion |
|---|---|---|---|
| | Formulation | Formulation | Formulation |
| | mg/bottle | mg/bottle | mg/bottle |
| | 0.65 mg/ml TCG | 0.325 mg/ml TCG | 0.10 mg/ml TCG |
| | concentration | concentration | concentration |
| | Composition | Composition | Composition |
| | 13.10 | 13.11 | 13.12 |
| Ticagrelor | 65 | 65 | 65 |
| Hydroxy | 8000 | 16000 | 16000 |
| Propyl | | | |
| beta | | | |
| cyclodextrin | | | |
| (HPβCD) | | | |
| Dextrose 5 | 100 ml | 200 ml | 650 ml |
| w/v % | | | |
| pH | 5.12 | 5.6 | 6.61 |
| Osmolarity | 512 | 425 | 336 |
| (mOsmol/kg) | | | |
| Clarity | Clear solution | Clear solution | Clear solution |

TABLE 28

| | RTU Infusion Formulation |
|---|---|
| | mg/bottle |
| | 2 mg/ml TCG concentration |
| | Composition 13.13 |
| Ticagrelor | 65 |
| Hydroxy Propyl beta | 3000 |
| cyclodextrin (HPβCD) | |
| Dextrose 5 w/v % | 30 ml |
| pH | 7.05 |
| Osmolarity | 674 |
| (mOsmol/kg) | |
| Clarity | Clear solution |

### Manufacturing Process for a Ready-to-Use Infusion Formulation

A manufacturing process to make the exemplified ready-to-use solutions was as follows. In all cases a solvent as mentioned is prepared and taken in a beaker and heated to 40° C., then HPβCD is added to obtain a clear solution under stirring. After this the active ingredient Ticagrelor is added at 40° C. under constant stirring until a clear solution is obtained. This solution is filtered through a 0.22-micron filter and filled aseptically in a sterile glass bottle or an infusion bag.

24 mg/ml to 350 mg/ml HPβCD was required to obtain a stable Ticagrelor solution ready for infusion. The amount of cyclodextrin required was depending upon the volume of the targeted infusion medium.

Ticagrelor is a an active ingredient that is insoluble in water. The more it is in a diluted aqueous solution, the more tendency it has to precipitate. A proportional increase in cyclodextrin was required as the dilution factor for ticagrelor increased, when going from 30 ml to 100 ml to 200 ml. However, for a 650 ml volume and higher an amount of 16 g of cyclodextrin was found sufficient to hold the ticagrelor in the aqueous solution.

Note that no organic co-solvent, surfactant or other solubilizer were used.

Alternative Preparation Method Starting from a Concentrated Ticagrelor Solution

It is possible to dilute 1 vial of 8 ml containing 65 mg/vial ticagrelor and about 3 g HPβCD with 25 ml 5 w/v % dextrose and obtain a clear solution with a final volume of 33 ml.

However, this was not possible with a 0.9 w/v % NaCl solution as diluent for the concentrated ticagrelor solution.

This is of importance in medical treatments where a concentrated ticagrelor aqueous composition would be mixed with another medicine. It can lead to precipitation of ticagrelor, rendering the combination product unsuitable for intravenous administration.

### Alternative Solubilizer for Ticagrelor

### Example 14

Aqueous ticagrelor solutions were prepared using vitamin E TPGS as solubilizer for ticagrelor. Water soluble vitamin E TPGS was added to water to obtain different concentrations of 2.5 and 5.0 and 10.0 w/v % aqueous vitamin E TPGS solutions. To these solutions, kept at a temperate of 45° C.+/−5° C., portions of ticagrelor were gradually added.

In the experiment summarized in Table 29, 3 mg ticagrelor portions were added step-by-step to a 50 ml aqueous vitamin E TPGS solution (1^{st} stage).

TABLE 29
Aqueous ticagrelor solutions with vitamin
E TPGS as solubilizer for ticagrelor

| | Weight ticagrelor | | Concentration |
|---|---|---|---|
| | added | Volume | (mg ticagrelor/ |
| Formulation | (mg) | (mL) | ml) |
| 2.5% vitamin E TPGS | 30 | 50 | 0.600 |
| 5.0% vitamin E TPGS | 30 | 50 | 0.600 |
| 10.0% vitamin E TPGS | 30 | 50 | 0.600 |

20 ml of each diluent were then used in a 2^{nd} stage study. To 20 ml of each diluent 10 mg ticagrelor portions were added. This was reduced to 5 mg when the dissolution was taking longer.

TABLE 30
Aqueous ticagrelor solutions with vitamin
E TPGS as solubilizer for ticagrelor

| | | Volume | |
|---|---|---|---|
| | Weight | (mL) of 0.6 mg/ | |
| | ticagrelor | ml ticagrelor | Concentration |
| | added | solution from | (mg ticagrelor/ |
| Formulation | (mg) | Table 29 | ml) |
| 2.5% vitamin E TPGS | 220 | 20 | 11,600 (232/20) |
| 5.0% vitamin E TPGS | 286 | 20 | 14,900 (298/20) |
| 10.0% vitamin E | 384 | 20 | 19,800 (396/20) |
| TPGS | | | |

The following was observed: 10 mg ticagrelor dissolved after 5-10 minutes.
2.5% vitamin E TPGS solution saturated at 11.6 mg/ml
5.0% vitamin E TPGS solution saturated at 14.9 mg/ml
10.0% vitamin E TPGS solution saturated at 19.8 mg/ml

The aqueous ticagrelor solutions obtained are stored at 40° C. and 75% Relative Humidity or at 25° C. and 60% Relative Humidity, for a period of at least 3 months.

### Solubility, but Inadequate Storage Stability

Polyethylene glycol can solubilized ticagrelor. However, polyethylene glycol was found sensitive to degradation and this led to impurities.

1. An aqueous ticagrelor solution,
comprising ticagrelor and a solubilizer for ticagrelor
with a storage stability of at least 3 months
at 40° C. and 75% Relative Humidity or
at 25° C. and 60% Relative Humidity;
wherein said solubilizer is a cyclodextrin or vitamin E TPGS.

2. An aqueous pharmaceutical ticagrelor solution according to claim 1, comprising
an inclusion complex of ticagrelor in a cyclodextrin,
wherein the aqueous pharmaceutical ticagrelor solution comprises
0.10-14.0 mg/ml ticagrelor and
20-100 mg/ml of cyclodextrin in a quantity for solubilization of the ticagrelor in the selected volume of aqueous pharmaceutical solution (solubilizer for ticagrelor),
wherein the composition has a pH between 5.5-9 endpoints included,
and the aqueous pharmaceutical solution has a volume of 25 to 1000 ml,
with a storage stability of at least 3 months at 25° C. and 60% Relative Humidity.

3. Aqueous pharmaceutical ticagrelor solution according to claim 2, wherein the osmolality is between 300-900 mOsm/kg.

4. (canceled)

5. (canceled)

6. (canceled)

7. (canceled)

8. (canceled)

9. (canceled)

10. A ready-to-use infusion container comprising a composition according to claim 2.

11. Ready-to-use infusion container according to claim 10, comprising 3000-16000 mg of cyclodextrin.

12. Ready-to-use infusion container according to claim 10, comprising 5 w/v % dextrose or 0.9 w/v % sodium chloride.

13. (canceled)

14. A method for the manufacturing of an aqueous pharmaceutical ticagrelor composition according to claim 2, comprising the steps of:
preparing an aqueous solution of pH 5.5-9 preferably comprising a buffering agent, more preferably comprising a phosphate buffer,
introducing a cyclodextrin amount for inclusion of a pre-determined amount of ticagrelor (solubilizer for ticagrelor),
adding the pre-determined amount of ticagrelor, thereby obtaining an inclusion complex of ticagrelor in cyclodextrin.

15. Method according to claim 14, wherein heating is applied prior to the addition of ticagrelor.

16. Method according to claim 14, wherein the ticagrelor has a D90 particle size below 10 micrometers when tested using Malvern mastersizer.

17. An aqueous ticagrelor composition according to claim 1 comprising ticagrelor as active ingredient,
characterized, in that the composition is a solution comprising
a water-soluble inclusion complex of ticagrelor in a cyclodextrin,
wherein the composition has a pH between 6 to 8;
with a storage stability of at least 3 months at 40° C. and 75% Relative Humidity.

18. An aqueous ticagrelor composition according to claim 17, wherein the osmolality is between 350-900 mOsm/kg.

19. Aqueous ticagrelor composition according to claim 17, wherein the cyclodextrin is hydroxypropyl-beta-cyclodextrin.

20. Aqueous ticagrelor composition according to claim 17, comprising 15-40% w/w hydroxypropyl-beta-cyclodextrin.

21. Aqueous ticagrelor according to claim 20, comprising 2-15 mg/ml ticagrelor.

22. (canceled)

23. (canceled)

24. (canceled)

25. (canceled)

26. Aqueous ticagrelor solution according to claim 17, consisting of
5-15 mg/ml ticagrelor,
15-40% w/w of a hydroxypropyl-beta-cyclodextrin,
5 mM-20 mM of phosphate buffer,
optionally including a tonicity modifier,
wherein the pH is between 5.5 and 8.

27. A method of treating a ticagrelor responsive medical condition, comprising the step of using an aqueous ticagrelor composition according to claim 15.

28. Method of treatment according to claim 27, wherein the composition is administered as an injection or infusion, nasal gastric fluid, or drink.

29. Method of treatment-according to claim 27, wherein the treatment is the treatment of acute coronary syndrome (ACS) or myocardial infarction (MI), ischemic stroke, transient ischemic attack (TIA) or for the reduction of platelet-tumor cell interactions in a patient in need thereof.

30. Method of treatment according to claim 27, for use in a dual antiplatelet therapy (DAPT), comprising acetyl salicylic acid or salts thereof as second active ingredient in addition to ticagrelor.

31. (canceled)

32. (canceled)

33. (canceled)

34. (canceled)

35. (canceled)

36. A method of treating acute coronary syndrome (ACS), myocardial infarction (MI), ischemic stroke, transient ischemic attack (TIA) or for the reduction of platelet-tumor cell interactions in a patient in need thereof; comprising the step of intravenously administrating an aqueous ticagrelor solution-, wherein the solution has a storage stability of at least 3 months in accelerated storage conditions at 40° C. and 75% Relative Humidity.

37. (canceled)

38. (canceled)
